(19) United States
(12) Patent Application Publication
(10) Pub. No.: US 2013/0280205 A1
(43) Pub. Date: Oct. 24, 2013

(54) Activators of SGK-1 for Use as Cardioprotective Agents

(71) Applicant: GEORGIA REGENTS UNIVERSITY, AUGUSTA, GA (US)

(72) Inventors: Mahmood Mozaffari, Martinez, GA (US); Babak Baban, Augusta, GA (US)

(21) Appl. No.: 13/740,889

(22) Filed: Jan. 14, 2013

Related U.S. Application Data
(60) Provisional application No. 61/586,517, filed on Jan. 13, 2012.

(51) Int. Cl. A61K 38/19; G01N 33/50

(52) U.S. Cl. CPC A61K 38/193; G01N 33/5008; A61K 38/191
USPC 424/85.1; 506/10

(57) ABSTRACT

Compositions for activating SGK-1 are provided. SGK-1 activators can be identified by the methods of screening provided herein. The SGK-1 activators can be used in the disclosed methods of reducing cell death and methods of treating ischemic-reperfusion injury. The ischemic-reperfusion injury can be due to a variety of complications resulting in blood loss to tissue and then the re-establishment of blood flow to the tissue.

### CROSS-REFERENCE TO RELATED APPLICATIONS

This application claims benefit of and priority to U.S. Provisional Application No. 61/586,517, which is incorporated by reference in their entirety.

### STATEMENT REGARDING FEDERALLY SPONSORED RESEARCH OR DEVELOPMENT

This invention was made with Support from American Heart Association, Southeast Affiliate, Grant Number 0755627B. The government has certain rights in the invention.

### FIELD OF THE INVENTION

This invention is generally directed to serum- and glucocorticoid-induced protein kinase 1 (SGK-1) activators and methods for treating ischemic-reperfusion with the SGK-1 activators.

### BACKGROUND OF THE INVENTION

Cardiovascular disease can result in restricted blood flow and reduced oxygen supply to several areas of the body resulting in ischemic injuries to various organs and tissues, including the brain, which can lead to stroke. After an ischemic event, it is necessary to restore the blood flow back to the deprived organs and tissues to prevent excessive cell death.

Reperfusion is the re-establishment of blood flow. Reperfusion leads to the re-oxygenation of the affected area following an ischemic episode and is critical for limiting irreversible damage to the affected tissue. However, reperfusion can also be damaging, such as an ischemic-reperfusion injury, which is caused by the restoration of coronary blood flow after an ischemic episode. The ischemic-reperfusion injury is caused by the generation and accumulation of reactive oxygen and nitrogen species during reperfusion. Up to 50% of the damage to the heart following a myocardial infarction can be due to reperfusion injury (Mellon, D. M., Hausenloy, D. J., Myocardial reperfusion injury. New England Journal of Medicine 2007, 357:1121).

One of the hallmarks of ischemia-reperfusion injury is an increase in cytosolic calcium levels, resulting from a depletion of oxygen during an ischemic event (Piper, H. M., Abdallah, C., Schafer, C., Annals of Thoracic Surgery 2003, 75:644; Yellon, D. M., Hausenloy, D. J., New England Journal of Medicine 2007, 357:1121). To date, treatments of patients with acute myocardial infarction with either an antagonist to block the influx of calcium or with a scavenger of the reactive oxygen species has each yielded disappointing clinical outcomes (Yellon, D. M., Hausenloy, D. J., New England Journal of Medicine 2007, 357:1121).

There is a need for new treatments of ischemic injury and ischemic-reperfusion injury.

Therefore it is an object of the invention to provide compositions and methods for inhibiting or reducing cell death due to ischemic, or ischemic-reperfusion injuries.

It is a further object of the invention to identify activators of SGK-1.

### SUMMARY OF THE INVENTION

Compositions and methods for treating or reducing ischemic injury or ischemic-reperfusion injury are provided. One embodiment provides a method for reducing cell injury resulting from an ischemic event or ischemic-reperfusion injury by increasing levels of phosphorylated SGK-1 in the cells of the injured tissue. A preferred method of increasing the levels of SGK-1 in cells includes, but is not limited to activating SGK-1 are provided. SGK-1 activation includes increasing protein levels of SGK-1, increasing level of SGK-1 phosphorylation or increasing the activity of SGK-1.

Primary reperfusion therapies, including primary percutaneous coronary intervention (PCI) and thrombolysis, are the standard of care for the treatment of acute coronary syndromes. Prompt restoration of blood flow to ischemic myocardium limits infarct size and reduces mortality. Paradoxically, however, the return of blood flow can also result in additional cardiac damage and complications, referred to as reperfusion injury. Such damage is more likely when reperfusion therapy is delayed. Thus, one embodiment provides a method of reducing or inhibiting ischemic-reperfusion injury due to reperfusion therapies by administering to the subject an effective amount of an SGK-1 activator prior to, during, after the reperfusion therapy or a combination thereof to increase the levels of phosphorylated SGK-1 in tissue receiving the reperfusion therapy or potentially damaged by the reperfusion therapy. Administering the SGK-1 activator to the subject before, during, or after reperfusion therapy can decrease the amount and severity of ischemic-reperfusion injury to the heart or blood vessels in the subject relative to the amount and severity of ischemic-reperfusion injury in subjects receiving reperfusion therapy without the administration of one or more SGK-1 activators.

Activators of SGK-1 include mineralocorticoids, gonadotropins, 1,25(OH)₂D₃, p53, cell-volume and hypotonic, cytokines such as GM-CSF and TNF-alpha, TGF-beta, serum, insulin, IGF-1, fibroblast- and platelet-derived growth factor, activators of the Erk signaling cascade and 12-O-tetradecanoylphorbol-13-acetate (TPA) or combinations thereof.

Another embodiment provides a method for treating ischemic-reperfusion injury by administering to a subject in need thereof an effective amount of a SGK-1 activator to increase cytosolic levels of phosphorylated SGK-1 protein relative to a control. The methods include administering one or more SGK-1 activators to a subject, preferably a human subject, in need thereof. The SGK-1 activator can directly or indirectly activate SGK-1 and decrease cell death due to ischemic-reperfusion.

Methods of screening or identifying SGK-1 activators are provided. The SGK-1 activators can directly or indirectly modulate SGK-1. Modulators of SGK-1 are useful for reducing ischemic-reperfusion injury induced cell death.

Also provided are pharmaceutical compositions containing a therapeutically effective amount of an SGK-1 activator and a pharmaceutically acceptable excipient, to treat one or more symptoms of ischemic-reperfusion injury.

### BRIEF DESCRIPTION OF THE DRAWINGS

FIG. 1 is a schematic diagram of an ischemic-reperfusion injury pathway. mTORC2: Mammalian target of rapamycin complex-2; SGK-1: serum-glucocorticoid-regulated kinase; GSK-3β: glycogen synthase kinase-3β; MPT: mitochondrial permeability transition.

FIGS. 2A and 2B are western blots showing total SGK-1, phopho-SGK-1 and actin. A represents normoxic heart and B represents ischemic-reperfused heart. β-actin was used as a loading control.

### DETAILED DESCRIPTION OF THE INVENTION

### A. Definitions

A compound is a chemical, biochemical or biological molecule, regardless of its size. The biological molecule, for example, can be a nucleic acid, protein or peptide.

Ischemic-reperfusion injury (“IR” injury) refers to an injury caused by the restoration of blood flow to an area of a tissue or organ that had previously experienced deficient blood flow due to an ischemic event. Ischemic-reperfusion injury can be caused naturally (e.g., restoration of blood flow following a myocardial infarction), a trauma, or by one or more surgical procedures or other therapeutic interventions that restore blood flow to a tissue or organ that has been subjected to a diminished supply of blood. Surgical procedures can include, but are not limited to, coronary artery bypass graft surgery, coronary angioplasty, and organ transplant surgery.

An SGK-1 activator is any compound that increases phosphorylation of SGK-1, increases protein levels of SGK-1 or increases the activity of SGK-1 proteins.

As defined herein, a “therapeutically effective amount” is an amount sufficient to achieve a desired therapeutic or prophylactic effect in a subject in need thereof under the conditions of administration, such as, for example, an amount sufficient to inhibit (e.g., prevent, delay) ischemia and ischemic-reperfusion injury in a subject (e.g., by reducing cell death in the affected tissue of the subject). The effectiveness of a therapy can be determined by suitable methods known by those of skill in the art.

The terms “treatment,” “treating,” or “treat” refer to any specific method or procedure used for the cure of, inhibition of, reduction of, prevention of, elimination of, or the amelioration of a disease or pathological condition (e.g., ischemic-reperfusion injury).

### B. Methods of Screening for SGK-1 Activators

The method of screening for SGK-1 activators can encompass screening for compounds that cause phosphorylation, increased activity, or increased expression of SGK-1. Increased phosphorylation levels of SGK-1 refers generally to the relative number of SGK-1 proteins that are phosphorylated in a cell rather than the degree of phosphorylation of a single SGK-1 protein. The selected compounds are useful as a therapeutic or research tools for the treatment and study of ischemic or ischemic-reperfusion injury.

The methods of screening for a compound that results in the phosphorylation of SGK-1 proteins in a cell, for example an endothelial or cardiac cell, can include contacting and SGK-1 protein with the test compound and determining the relative amounts of phosphorylated SGK-1 proteins.

Alternatively, compounds that increase the bioavailability or genetic expression of SGK-1 can also be screened. In this case, the test compound is administered to cell culture capable of expressing functional SGK-1. After administration of test compound, the cell culture can be screened for levels of SGK-1 RNA or SGK-1 protein. Compounds that increase SGK-1 RNA or SGK-1 protein levels are selected. Detecting activated SGK-1 can be performed with any of the standard techniques in the art for detecting phosphorylated SGK-1, increased expression levels of SGK-1 protein or SGK-1 protein activity.

The screening methods can also include bringing into contact a test compound and a cell that contains the SGK-1 gene and/or protein. The test compound is a SGK-1 activator if there is an increase in phosphorylated SGK-1, expression levels of SGK-1 or SGK-1 activity.

The screening methods can be performed in the presence and absence of the test compound wherein an increase in activated SGK-1 in the presence of the test compound compared to the in the absence of the test compound is indicative that the test compound is an SGK-1 activator.

1. SGK-1

Serum glucocorticoid regulated kinases are a family of serine/threonine protein kinases that include SGK-1, SGK-2, and SGK-3. SGK-1 is expressed in virtually all tissues which have been tested to date, but the amounts of expressed mRNA vary widely depending on the nature of the tissue type investigated (Gonzalez-Robayna et al., Mol Endocrinol 13:1318-1337, 1999; Waldegger et al., Gastroenterology 116:1081-1088, 1999; Alliston et al., Endocrinology 141:385-395, 2000; Klingel et al., Am J Physiol Gastrointest Liver Physiol 279:G998-G1002, 2000; Loffing et al., Am J Physiol Renal Physiol 280:F675-F682, 2001).

The active form of SGK-1 requires activation by phosphorylation. This can be mediated by a signaling cascade in which phosphatidylinositol (PI)-3 kinase and the 3-phosphoinositide-dependent kinases PDK1 and PDK2 are involved. SGK-1 has two known phosphorylation sites, Thr256 and Ser422. One or both of these sites can be phosphorylated for SGK-1 to be activated. Thus, one embodiment provides activators of SGK-1 by activating the PI-3 kinase pathway.

One downstream pathway of SGK-1 involves GSK3β. GSK3β is a critical regulator of cell death. Phosphorylation of GSK3β results in an inactive form and reduces cell death. SGK-1 phosphorylates GSK3β, rendering it inactive and thus reducing cell death.

2. Test Compound

Candidate compounds for screening according to the methods herein include any chemical compounds, including libraries of chemical compounds. There are a number of different libraries used for the identification of small molecule modulators, such as activators, including: (1) chemical libraries, (2) natural product libraries, and (3) combinatorial libraries comprised of random peptides, oligonucleotides or organic molecules. Chemical libraries consist of random chemical structures, or analogs of known compounds, or analogs of compounds that have been identified as “hits” or “leads” in prior drug discovery screens, some of which may be derived from natural products or from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of SGK-1 activators through use of the various libraries described herein permits modification of the candidate compound to optimize the capacity of the compound to activate SGK-1.

### C. Methods of Activating SGK-1

SGK-1 activators can directly or indirectly activate SGK-1. SGK-1 can be activated directly with a compound that phosphorylates SGK-1, increases levels of SGK-1 or increases SGK-1 activity. This can include a kinase or a transcription activator. SGK-1 can be indirectly activated with a compound that modulates a factor upstream of the SGK-1 pathway but does not act directly on SGK-1 or the SGK-1 gene product.

Another method for activating SGK-1 can include blocking or inhibiting the agent responsible for causing a decrease in phosphorylation of SGK-1, a decrease in expression of SGK-1, or a decrease in activity of SGK-1. For example, phosphorylated SGK-1 is decreased during Ischemic-reperfusion injury therefore blocking or inhibiting the mechanism causing the decreased SGK-1 phosphorylation can be considered an SGK-1 activator.

1. SGK Activator

SGK-1 activators can increase transcription levels of SGK-1, protein levels of SGK-1, phosphorylation of SGK-1 or activity of SGK-1.

There are many compounds known to activate the transcription of SGK-1. These include mineralocorticoids (Brennan et al., Mol Cell Endocrinol 166:129-136, 2000; Shigaev et al., Am J Physiol Renal Physiol 278:F613-F619, 2000; Bhargava et al., Endocrinology 142:1587-1594, 2001), gonadotropins (Gonzalez-Robayna et al., Mol Endocrinol 14:1283-1300, 2000), 1,25(OH)2D₃ (Akutsu et al., Mol Endocrinol 15:1127-1139, 2001), p53, cell-volume and hypotonic changes (Waldegger et al., PNAS 94:4440-4445, 1997; Klingel et al., 2000; Waldegger et al., Cell Physiol Biochem 10:203-208, 2000; Rozansky et al., Am J Physiol Renal Physiol 283:F105-F113, 2002; Wamtges et al., Pflugers Arch 443:617-624, 2002), cytokines such as GM-CSF and TNF-alpha (Cooper et al., J Bone Miner Res 16:1037-1044, 2001, Cowling et al. J Leukoc Biol 7:240-248, 2000) or by TGF-beta (Kumar et al., J Am Soc Nephrol 10:2488-2494, 1999; Waldegger et al., Gastroenterology 116:1081-1088, 1999). Induction of SGK takes place in further growth-dependent signaling pathways by serum (Webster et al., Mol Cell Biol 13:2031-2040, 1993), insulin and IGF-1 (Kobayashi et at, Biochem J 339:319-328, 1999; Park et al., EMBO J. 18:3024-3033, 1999; Perrotti et al., J Biol Chem 276:9406-9412, 2001), FSH (Alliston et al., Mol Endocrinol 11:1934-1949, 1997), fibroblast- and platelet-derived growth factor, activators of the Erk signaling cascade and TPA (Mizuno et al., Genes Cells 6:261-268, 2001). The increase of levels of SGK-1 results in more SGK-1 available to be phosphorylated and thus, activated.

The activator can be SGK-1 itself because the addition of SGK-1 increases the levels of SGK-1.

The SGK-1 activator can be a molecule that acts upstream of SGK-1, such as P13-kinase. IL-6 can activate p38 MAPK and overexpression of p38 MAPK can increase phosphorylation of SGK (Meng et al. Am J Physiol Cell Physiol 289:C971-C981, 2005). β-catenin has also been shown to upregulate SGK-1 (Wang et al. Cell Physiol Biochem 2010). PDK1 agonists can be used as SGK-1 activators (Kobayashi et al. Biochem J. 339:319-328, 1999). mTOR-Raptor can activate SGK-1 (Hong et al. Mol Cell 30:701-711, 2008).

2. Detecting Activated SGK-1.

Activated SGK-1 can be identified by detecting the phosphorylation, increased activity or increased expression levels of SGK-1. Assays for each of these detection methods are well known in the art. For example, a western blot using an antibody for detecting phosphorylated SGK-1 can be used to detect phosphorylation. The activity of SGK-1 can be tested using an activity assay protocol from Signal Chem. And increased levels of expression can be determined with a northern blot or western blot. The increased expression levels can be determined by an increase in RNA or an increase in protein.

### D. Methods of Diagnosing

Methods for diagnosing the propensity of developing or the severity of ischemic or ischemic-reperfusion injury include detecting the level of phosphorylated SGK-1 in cardiac or endothelial tissue. A reduced level of phosphorylated SGK-protein is indicative of cellular injury or an increased risk of developing cellular injury including cell death.

The present studies determined that phosphorylated SGK-1 levels are markedly lowered in response to an ischemic-reperfusion injury. The absence of activated SGK-1, for example phosphorylated SGK-1 protein, in sample from a subject is indicative that the subject has or is likely to develop ischemic or ischemic-reperfusion injury including cell death.

The presence of activated SGK-1 in a sample from a subject can be detected by any standard techniques known in the art. The detection of activated SGK-1 can entail detecting phosphorylated SGK-1, increased expression levels of SGK-1 or SGK-1 activity.

The disclosed method system can further involve the use of a computer system to compare levels of the activated SGK-1 to control values. For example, the computer system can use an algorithm to compare levels of two or more biomarkers and provide a score representing the risk of ischemic-reperfusion injury based on detected differences.

Also provided is an apparatus for use in predicting the ischemic-reperfusion injury in a subject that includes an input means for entering activated SGK-1 level values from a sample of the subject, a processor means for comparing the values to control values, an algorithm for giving weight to specified parameters, and an output means for giving a score representing the risk of cell death.

1. Sample

The sample used to detect the presence or absence of activated SGK-1 can be a biological sample. The sample can be, but is not limited to, blood, cells, or tissue. The detection of activated SGK-1 in a sample can be done in vitro.

2. Subject

The subject from which a sample is taken for testing for the presence or absence of activated SGK-1 can be a mammal, preferably a human. The subject can be healthy or can have or is suspected to have heart disease. The subject can be someone who is about to suffer an ischemic event, is currently undergoing an ischemic event or an ischemic-reperfusion event, or has already endured an ischemic event or ischemic-reperfusion injury.

### E. Method of Reducing Cell Death

SGK-1 activators can be used in methods of reducing cell death due to ischemia or ischemic-reperfusion, in particular endolethial cell death and cardiac cell death. For example, activated SGK-1 is responsible for phosphorylating GSK3β which in turn reduces cell death.

In one embodiment, a reduction in cell death in the presence of an SGK-1 activator can be determined by comparing it to the amount of cell death in the absence of SGK-1 activator.

The cells affected by SGK-1 activators can be any cell type in which the cell or the tissue the cell is in can be affected by ischemic-reperfusion injury. For example, cardiomyocytes are often damaged during ischemic-reperfusion injury. Thus, one embodiment provides a method for reducing injury to cardiomyocytes by administering an effective amount of a SGK-1 activator to a subject in need thereof to increase the level of phosphorylated SGK-1 in the cardiomyocytes relative to the levels of phosphorylated SGK-1 in the cardiomyocytes prior to administration of the SGK-1 activator.

Still another embodiment provides a method of reducing injury to smooth muscle cells due to ischemia or ischemic-reperfusion by administering to the smooth muscle cells an effective amount of an SGK-1 activator to increase the levels of phosphorylated SGK-1 protein in the smooth muscle cells.

### F. Methods of Treating Ischemic-Reperfusion Injury

Disclosed herein are methods of treating or preventing an ischemic-reperfusion injury in a subject by administering to the subject a therapeutically effective amount of at least one SGK-1 activator. Preventing ischemic-reperfusion injury can include reducing the risk of injury.

The methods of treating can be done by targeting the SGK-1 activator to a specific site. For example, targeting SGK-1 activators specifically to cardiomyocytes or to the vascular endothelium could increase the effectiveness of the treatment. Peptides, such as CSTSMLKAC (SEQ ID NO:1), CKPGTSSYC (SEQ ID NO:2) and CPDRSVNNC (SEQ ID NO:3), that target ischemic myocardium can be used as a targeting agent to direct the SGK-1 activator to the specific site of ischemic-reperfusion injury (Kanki et al. J of Molecular and Cellular Cardiology. 50(5):841-848, 2011). The targeting agents can be peptides, proteins, antibodies, nucleic acids, nanoparticles, or small molecules.

One embodiment provides a method of reducing or inhibiting ischemic-reperfusion injury due to reperfusion therapies by administering to the subject an effective amount of an SGK-1 activator prior to, during, after the reperfusion therapy or a combination thereof to increase the levels of phosphorylated SGK-1 in tissue receiving the reperfusion therapy or potentially damaged by the reperfusion therapy. Administering the SGK-1 activator to the subject before, during, or after reperfusion therapy can decrease the amount and severity of ischemic-reperfusion injury to the heart or blood vessels in the subject relative to the amount and severity of ischemic-reperfusion injury in subjects receiving reperfusion therapy without the administration of one or more SGK-1 activators.

Activators of SGK-1 include mineralocorticoids, gonadotropins, 1,25(OH)2D₃, p53, cell-volume and hypotonic, cytokines such as GM-CSF and TNF-alpha, TGF-beta, serum, insulin, IGF-1, fibroblast- and platelet-derived growth factor, activators of the Erk signaling cascade and 12-O-tetradecanoylphorbol-13-acetate (TPA).

Surgical equipment and implants can be coated with a composition containing an SGK-1 activator. For example, stents used for heart conditions can be coated with an SGK-1 activator and thus providing the activator close to or directly in the tissue being damaged from ischemic-reperfusion.

The subjects to be treated with these methods are any subject who has suffered or is at risk of suffering ischemic-reperfusion. In particular those subjects who have suffered myocardial infarction or any ischemic-reperfusion stemming from some type of heart disease.

Any of the SGK-1 activators disclosed herein can be used in these methods.

1. Ischemic-Reperfusion Injury

Ischemic-reperfusion injury can occur in several organs or tissues in the body. Examples of the organs affected by ischemic-reperfusion injury, include but are not limited to, one or more of gastrointestinal tract, liver, lung, kidney, heart, brain, spinal cord, and crushed limb.

In a preferred embodiment, the heart is affected by ischemia-reperfusion. This can be caused by many heart conditions or surgical procedures.

2. Combination Therapies

The SGK-1 activator can be administered alone or in combination with at least one other therapeutic agent or treatment or example primary reperfusion. The other therapeutic treatment can be an anti-inflammatory agent, immune modulators, ischemic preconditioning, hypothermia, hydrogen sulfide, selectin agonists and antagonists, cytoprotective kinases (e.g. insulin receptor kinase, insulin-like growth factor 1 receptor kinase, Src kinase, Akt kinase), or adenosine receptor agonist or protein kinase C activator and a complement inhibitor.

For combination therapies, the SGK-1 activator can be co-administered with another therapeutic agent. Co-administration involves the administration of two or more agents to an animal so that both agents and/or their metabolites are present in the animal at the same time. Co-administration includes simultaneous administration in separate compositions, administration at different times in separate compositions, or administration in a composition in which both agents are present.

3. Administration

The disclosed compositions can be administered in any suitable manner. The manner of administration can be chosen based on, for example, whether local or systemic treatment is desired, on the area to be treated, and on what type of composition is being delivered (e.g., peptide, nucleic acid, etc.). For example, the compositions can be administered orally, parenterally (e.g., intravenous, subcutaneous, or intramuscular injection), or the like.

For oral administration, solid dosage forms of tablets, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The peptides can also be in micro-encapsulated form, if appropriate, with one or more excipients.

Peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. For example, PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine.

To ensure full gastric resistance a coating can be impermeable to at least pH 5.0. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

To aid dissolution of peptides into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of peptides are for instance the fatty acids oleic acid, linoleic acid and linolenic acid.

Controlled release oral formulations may be desirable. The peptides could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The peptides could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Parenteral administration of the composition is generally characterized by injection. Injectables can be prepared in conventional forms, either as liquid solutions or suspensions, solid forms suitable for solution of suspension in liquid prior to injection, or as emulsions. Parenteral administration can involve the use of a slow release or sustained release system such that a constant dosage is maintained.

The compositions can also be administered intranasally. As used herein, “topical intranasal administration” means delivery of the compositions into the nose and nasal passages through one or both of the nares and can involve delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

The compositions can be delivered via a catheter or similar device that allows for direct administration of the composition to the area of interest, such as the heart.

i. Dosage

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art.

Dosage regimens are adjusted to provide the optimum desired response (e.g., effective amount or a therapeutic amount). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Guidance can be found in the literature for appropriate dosages for given classes of pharmaceutical products.

For example, a typical daily dosage of a composition having a peptide used alone might range from about 1 μg/kg to up to 100 mg/kg of body weight or more per day, depending on the factors mentioned above. For example dosages can be about 0.01 to 5 mg/kg of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg body weight.

Dosage can vary, and can be administered in one or more dose administrations daily, for one or several days. An exemplary treatment regime entails administration twice per day, once per day, once per week, once every two weeks, once every three weeks, once every four weeks, once a month, once every 3 months or once every three to 6 months.

The compositions disclosed herein can be administered prophylactically to patients or subjects who are at risk for suffering ischemic-reperfusion injury or are currently suffering ischemic-reperfusion, or therapeutically to patients who have undergone ischemic-reperfusion.

ii. Timing

The timing for administering the composition can vary. If the ischemic event, which leads to Ischemic-reperfusion injury, is anticipated as in organ transplantation, then the compositions described herein can be administered prophylactically, prior to the operation or ischemic event. For the treatment of ischemic-reperfusion injuries caused by therapeutic interventions, such as surgical procedures, it is preferable that the pharmaceutical composition is administered to a subject undergoing treatment prior to the therapeutic intervention (e.g., cardiac surgery, organ transplant). For example, a composition can be administered to a subject undergoing treatment, e.g., about 1 hour, about 2 hours, about 3 hours, about 4 hours, about 5 hours, about 12 hours, about 24 hours, or about 48 hours prior to the therapeutic intervention. A composition can also be administered to a subject undergoing treatment, for example, about 5 minutes, about 10 minutes, about 15 minutes, about 20 minutes, about 30 minutes or about 45 minutes prior to the therapeutic intervention.

Alternatively, or in addition, a composition can be administered at the time of or during the therapeutic intervention. For example, the composition can be administered one or more times during the course of a therapeutic intervention in intervals (e.g., 15 minute intervals). Alternatively, a composition can be administered continuously throughout the duration of a therapeutic intervention.

In certain instances, the composition can be administered to a subject undergoing treatment after a therapeutic intervention. For example, a composition can be administered to a subject, e.g., about 1 hour, about 2 hours, about 3 hours, about 4 hours, about 5 hours, about 12 hours, about 24 hours, or about 48 hours after the therapeutic intervention. A composition can also be administered to a subject about 5 minutes, about 10 minutes, about 15 minutes, about 20 minutes, about 30 minutes or about 45 minutes after the therapeutic intervention.

In circumstances, such as myocardial infarction, wherein the subject is not aware an Ischemic-reperfusion injury is about to occur, the composition can be administered after the Ischemic-reperfusion injury. Preferably, the composition is administered within minutes of the ischemic-reperfusion injury but can also be administered within the first 24 hours of Ischemic-reperfusion injury. In some instances, the composition can be administered up to 36, 48, or 72 hours after Ischemic-reperfusion injury.

In some instances, chronic ischemic injury occurs, chronic prophylactic treatment can be necessary to prevent cell death.

iii. Additional Ingredients

The compositions disclosed herein can be administered to a subject along with additional ingredients. As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Additional ingredients can also include a targeting agent wherein the SGK-1 activator is targeted to specific cells or tissue in the body. For example, SGK-1 activators can be encapsulated within, dispersed in, associated with, or conjugated to a nanoparticle functionalized with one or more targeting agents.

The nanoparticles may be formed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. In some cases, the mitochondrial targeting agents are polypeptides that are covalently linked to the surface of the nanoparticle after particle formulation. In other cases, the mitochondrial targeting agents are lipophilic cations that are covalently bound to the particle surface. In some cases, a cationic polymer is incorporated into the particle to target the particle to the mitochondrion.

### G. Pharmaceutical Compositions

The disclosed compositions can be administered in vivo either alone or in a pharmaceutically acceptable carrier. By “pharmaceutically acceptable” is meant a material that is not biologically or otherwise undesirable, i.e., the material can be administered to a subject, along with the composition disclosed herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into nanoparticles, microparticles, liposomes, or cells).

The pharmaceutical compositions can be prepared in accordance with standard procedures and are administered at dosages that are selected to reduce, prevent, or eliminate, or to slow or halt the progression of, the

1. Pharmaceutically Acceptable Carriers

The compositions disclosed herein can be used prophylactically and therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

a. Liposomes

Pharmaceutical composition having an effective amount of one or more peptides can be carried in a liposome. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 .mu.m. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and are made of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 .mu.m. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 .mu.m, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the disclosed compositions into liposomes. Generally, the fatty acid is a polar lipid. Thus, the fatty acid can be a phospholipid. The provided compositions can include either natural or synthetic phospholipid. The phospholipids can be selected from phospholipids containing saturated or unsaturated mono or disubstituted fatty acids and combinations thereof. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can include palmitoyl 16:0.

b. Nanoparticles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

The nanoparticles can carry the SGK-1 activator. In some instances, the nanoparticle can be coated with a targeting agent that targets ischemic tissue.

### Examples

### I. Activators of SGK-1 as Cardioprotective Agents

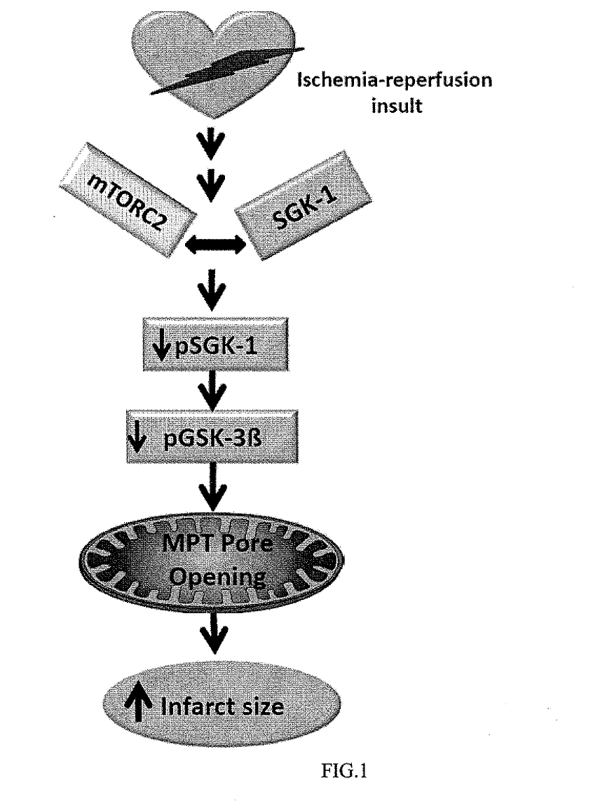

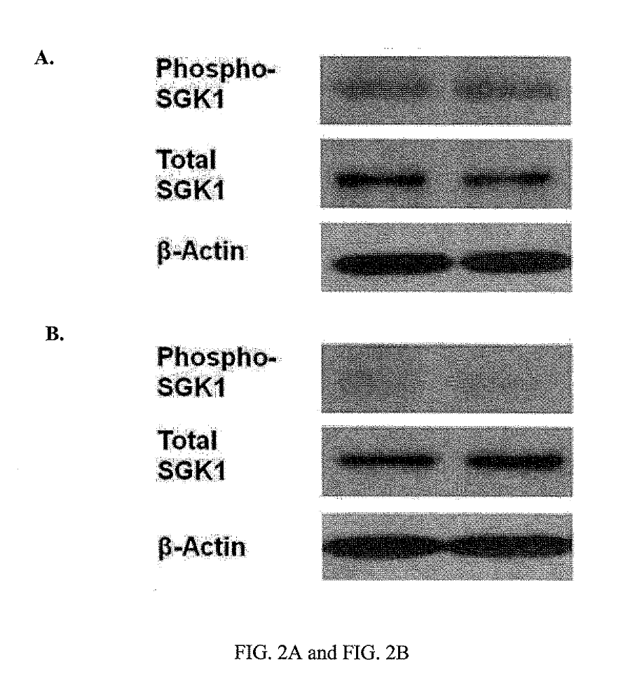

Ischemic-reperfusion can lead to a decrease in phosphorylated SGK-1 and ultimately an increase in infarct size. The schematic diagram in FIG. 1 shows the pathway starting from ischemic-reperfusion injury and resulting in increased infarct size. Studies showed that in normoxic hearts, there were normal levels of phosphorylated SGK-1 (FIG. 2A). In ischemic-reperfused hearts, there was a decrease in phosphorylated SGK-1 compared to the normoxic hearts (FIG. 2B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for reducing or inhibiting ischemic or ischemic-reperfusion injury in a subject comprising administering to the subject an effective amount of an SGK-1 activator to increase the levels of phosphorylated SGK-1 in endothelial or cardiac tissue of the subject.

2. The method of claim 1, wherein the SGK-1 activator is selected from the group consisting of mineralocorticoids, gonadotropins, 1,25(OH)2D₃, p53, cell-volume and hypotonic, cytokines such as GM-CSF and TNF-alpha, TGF-beta, serum, insulin, IGF-1, fibroblast- and platelet-derived growth factor, activators of the Erk signaling cascade, 12-O-tetradecanoylphorbol-13-acetate (TPA), and combinations thereof.

3. The method of claim 1, wherein the ischemic or ischemic-reperfusion injury is due to primary reperfusion therapy.

4. The method of claim 1, wherein the ischemic or ischemic-reperfusion injury results from myocardial infarction.

5. A method for screening for activators of SGK-1 comprising determining the ability of a test compound to modulate the expression, phosphorylation or activity of SGK-1.

6. The method of claim 5 further comprising
a) contacting the test compound to SGK-1; and
b) measuring the expression, phosphorylation or activity of SGK-1, wherein a compound that increases expression, phosphorylation or activity of SGK-1 is identified as an SGK-1 activator.

7. A method of reducing cell death comprising contacting an injured or diseased tissue with a therapeutically effective amount of an SGK-1 activator.

8. The method of claim 7, wherein the cell death is reduced in the presence of an SGK-1 activator compared to in the absence of an SGK-1 activator.

9. The method of claim 7, wherein the cell is a cardiomyocyte.

10. The method of claim 7, wherein the tissue has ischemic-reperfusion injury.

11. A method of preventing or treating an ischemic-reperfusion injury comprising administering to a subject a composition comprising a therapeutically effective amount of an SGK-1 activator following myocardial infarction or primary reperfusion therapy.

12. The method of claim 11, wherein the composition further comprises a pharmaceutically acceptable excipient.

13. The method of claim 11, wherein the subject has suffered a myocardial infarction.

14. A pharmaceutical composition comprising a therapeutically effective amount of an SGK-1 activator to increase levels of phosphorylated SGK-1 in endothelial or cardiac tissue of a subject and a pharmaceutically acceptable excipient.


SEQUENCE LISTING (3 sequences)

SEQ ID NO: 1 - protein, 9 aa, Artificial Sequence
  Synthetic peptide homologous to human ischemic myocardium
CSTSMLKAC

SEQ ID NO: 2 - protein, 9 aa, artificial sequence
  Synthetic peptide homologous to human ischemic myocardium
CKPGTSSYC

SEQ ID NO: 3 - protein, 9 aa, artificial sequence
  Synthetic peptide homologous to human ischemic myocardium
CPDRSVNNC